(19) United States
(12) Patent Application Publication
(10) Pub. No.: US 2020/0297205 A1
(43) Pub. Date: Sep. 24, 2020

(54) TOOTHIE STICK

(71) Applicant: Candid Care Co., New York, NY (US)

(72) Inventors: Sam Hill, Stockton, NJ (US); Peter MacRobert, New York, NY (US); Mary Titus, New York, NY (US)

(73) Assignee: Candid Care Co., New York, NY (US)

(21) Appl. No.: 16/822,895

(22) Filed: Mar. 18, 2020

Related U.S. Application Data
(60) Provisional application No. 62/820,568, filed on Mar. 19, 2019.

(51) Int. Cl. A61B 1/24; A61B 1/04

(52) U.S. Cl. CPC A61B 1/24; A61B 1/04

(57) ABSTRACT

A camera stick comprises a dental component for being positioned in a subject's mouth, a holder for holding a camera device, and at least one coupling mechanism mechanically coupled to the dental component and holder. The coupling mechanism is adjustable for enabling the dental component and holder to be positioned at a selected relative position/orientation. The dental component includes a cheek stretcher. A method of operating the camera stick comprises setting a ratcheting mechanism of the coupling mechanism to a predetermined position, inserting the cheek stretcher into a subject's mouth, and taking a picture of a predetermined part of the subject's mouth using the camera device. Before taking the picture, the handle can be positioned relative to the cheek stretcher to cause the predetermined part of the subject's mouth to be exposed to the camera device. A method of forming the camera stick also is provided.

### CROSS REFERENCE TO RELATED APPLICATION

This application claims the benefit of priority of U.S. Provisional Application No. 62/820,568, filed Mar. 19, 2019, which is hereby incorporated by reference as if set forth fully herein.

### BACKGROUND

Dental aligners are orthodontic devices that typically are used to adjust teeth. Dental aligners are orthodontic appliances typically formed of transparent plastic. In order for a manufacturer to craft dental aligners, photographs of a patient's teeth often are employed to help in the crafting. Oftentimes such photographs are taken by the patients themselves using a handheld image capturing device such as, for example, smartphone cameras, and are uploaded electronically or otherwise provided to the manufacturer. However, patients typically have a great deal of uncertainty with respect to how the device should be positioned in order to capture an image properly. For example, patients often do not know the correct orientation, rotation, angle, and distance at which to hold their device when taking a photograph. Additionally, for technical reasons, rear cameras may have to be used, creating an additional barrier to taking the photographs successfully. As a result of the foregoing limitations, when images are captured, their quality often is sub-par, resulting in the photographs being rejected by the dental aligner manufacturer (e.g., as high as a 65% rejection rate).

U.S. Patent Application Pub. No. 20180303331 A1 discloses an imaging device including a support, a dental retractor fastened to the support and defining a retractor opening, a mirror fastened to at least one of the support and the retractor, and a fastening structure. The fastening structure fastens an image acquisition apparatus to the support in a position in which the acquisition apparatus is oriented, in order to receive a composite image including a direct image of the retractor opening and an image of the retractor opening reflected by the mirror.

Canadian Patent No. CA 2375007 C is directed to a dental camera for obtaining a picture of an object in the mouth of a patient. The dental camera includes a light source for irradiating the object, a reflected light receipt element for receiving the light reflected from the object, such as one or more teeth and a mouthpiece. The mouthpiece has an opening which permits the passage of light to the reflected light receipt element. The reflected light passes through the mouthpiece and is received by the reflected light receipt element. The light of the light source preferably passes through the mouthpiece.

Smile Lite MDP is purported to be a “mini photo studio” equipped with three groups of light emitting diodes. Each group of diodes can be lit up individually, and the illumination power is controllable using a dimmer.

Smile Lite MDP is purported to be a plug and play device that does not need a special application or calibration process. See https://www.smileline.ch/en/products/smile-lite-mdp (last accessed on Feb. 27, 2019).

Smartphone Dental Light (SDL) by PhotoMed is a dental accessory for a smartphone. The SDL has two High CRI 95+ LED panels that have 8 levels of brightness. A lowest one of the brightness levels is purported to be ideal for dental photography. Higher power levels are purported to be for video taken further from a subject. The LED panels have swivel hinges that enable positioning of the hinges for different illumination types. Phones can be attached using a universal mount, and a grip includes a bluetooth trigger for functioning as a grip for one-handed photography. The SDL also has a case, a charger, and charging cable to charge the lights from one charger. See https://www.photomed.net/sdl.htm (last accessed on Feb. 27, 2019).

Non-dental selfie sticks also are known.

### SUMMARY

The above and other limitations are overcome by a camera stick, a method of forming the camera stick, and a method of operating the camera stick, according to example aspects herein. In accordance with an example embodiment herein, the camera stick comprises a dental component for being positioned in a subject's mouth, a holder for holding a camera device, and at least one coupling mechanism mechanically coupled to the dental component and the holder. The at least one coupling mechanism is adjustable for enabling the dental component and holder to be positioned in space at a selected position and orientation relative to one another.

In accordance with one example embodiment herein, at least one of the holder and the dental component is mechanically rotatably coupled to the coupling mechanism.

Also, in one example embodiment herein, the at least one coupling mechanism comprises a first coupling mechanism, a second coupling mechanism, and a ratcheting mechanism interposed between and mechanically coupled to the first and second coupling mechanisms. The ratcheting mechanism enables at least one of the first and second coupling mechanisms to be rotated relative to another one of the first and second coupling mechanisms. Also in one example embodiment herein, the dental component comprises a cheek stretcher, and the camera stick further comprises a connecting component mechanically coupling the cheek stretcher to an end of the first coupling mechanism. Preferably, the connecting component has a base, the cheek stretcher is rotatably mechanically coupled to the base, and the connecting component is mechanically rotatably coupled to the end of the first coupling mechanism.

In still an additional example embodiment herein, the cheek stretcher comprises a first mouthpiece and a second mouthpiece, each being curved in shape and including an annular groove for receiving the subject's mouth. The cheek stretcher enables the subject's mouth to be opened to an extent that enables at least a substantial portion of a bite and/or teeth of the subject's mouth to be exposed to the camera device for being captured in an image.

In a further example embodiment herein, the ratcheting mechanism comprises an upper portion and a lower portion, the upper portion includes an upper surface and a lower surface, the lower portion also includes an upper surface and a lower surface, and the upper surface of the lower portion is engaged with the lower surface of the upper portion. In one example, at least one of the upper surface or the lower surface of at least one of the upper portion or the lower portion forms a teethed surface, and the upper portion and lower portion can be rotatably engaged with one another.

The first coupling mechanism is rotatably mechanically coupled to the upper portion, and the second coupling mechanism is rotatably mechanically coupled to the lower portion. At least one of the first coupling mechanism or the second coupling mechanism is rotatable around an axis extending along a length of the at least one of the first coupling mechanism or the second coupling mechanism.

In still a further example embodiment herein, the camera stick also comprises a handle mechanically coupled to the ratcheting mechanism. In one example, the handle includes an elongate portion and a spindle attached thereto, the spindle being insertable into a bore hole of the ratcheting mechanism.

According to another example embodiment herein, the method of forming a camera stick is provided. The method comprises inserting a handle into an opening of a lower portion of a ratcheting mechanism, the lower portion being mechanically coupled to a first end of a first coupling mechanism. A next step includes engaging an upper portion of the ratcheting mechanism with the lower portion, the upper portion being coupled to a holder for holding a camera device. A further step includes coupling a cheek stretcher to a second end of the first coupling mechanism.

According to a further example embodiment herein, the method of operating a camera stick is provided. The method comprises setting a ratcheting mechanism of the camera stick to a predetermined position. The camera stick includes the ratcheting mechanism, a cheek stretcher coupled to the ratcheting mechanism by way of a first coupling mechanism, a camera holder coupled to the ratcheting mechanism by way of a second coupling mechanism, and a handle coupled to a lower surface of the ratcheting mechanism. The method also includes inserting the cheek stretcher into a subject's mouth, and operating a camera device held by the camera holder to take a picture of a predetermined part of the subject's mouth. Additionally, the method can include positioning the handle relative to the cheek stretcher to cause the predetermined part of the subject's mouth to be exposed to a view of the camera device. The predetermined part of the subject's mouth can include, by example and without limitation, at least one of a right bite, left bite, front bite, lower teeth, or upper teeth, of the subject's mouth.

### BRIEF DESCRIPTION OF THE DRAWINGS

FIG. 1 is a perspective view of a Toothie Stick device constructed according to an example aspect herein, wherein the Toothie Stick is represented holding an image capturing device.

FIG. 2 shows a ratcheting mechanism and parts of first and second coupling mechanisms of the Toothie Stick of FIG. 1.

FIG. 3 shows a holder, coupler, and part of a rod of the Toothie Stick of FIG. 1.

FIG. 4 shows a perspective view of part of the Toothie Stick of FIG. 1, holding the imaging capturing device.

FIG. 5 shows a rod of a second coupling mechanism, and couplers, of the Toothie Stick of FIG. 1.

FIG. 6 shows part of the Toothie Stick of FIG. 1, including a cheek stretcher, connecting component, first coupling mechanism, and part of the ratcheting mechanism.

FIG. 7 shows a perspective view of the Toothie Stick of FIG. 1, with a handle thereof removed, and positioned such that the image capturing device and cheek stretcher have a particular relative orientation.

FIG. 8 shows a perspective view of the part of the Toothie Stick shown in FIG. 7, positioned such that the image capturing device and cheek stretcher have another particular relative orientation.

FIG. 9 shows a holder, coupler, and part of a rod of the Toothie Stick of FIG. 1, wherein the image capturing device is shown held by the holder.

FIG. 10 shows a perspective view of the holder of the Toothie Stick of FIG. 1, removed from the Toothie Stick.

FIG. 11 shows a method for assembling a Toothie Stick, according to an example aspect herein.

FIG. 12 represents assembly of the Toothie Stick according to the method of FIG. 11, in an example embodiment herein.

FIG. 13 shows a method for using a Toothie Stick to capture images of bites and teeth of a subject's mouth, according to an example aspect herein.

Same reference numerals appearing in different ones of the figures represent the same components, although each component may not be described in detail herein with respect to the description of each separate figure.

### DETAILED DESCRIPTION

The inventors have developed a device and methods that standardize the manner in which patients and users can take photographs of their teeth and mouths, to ensure greater accuracy and quality in taken photographs to be used for creating dental aligners.

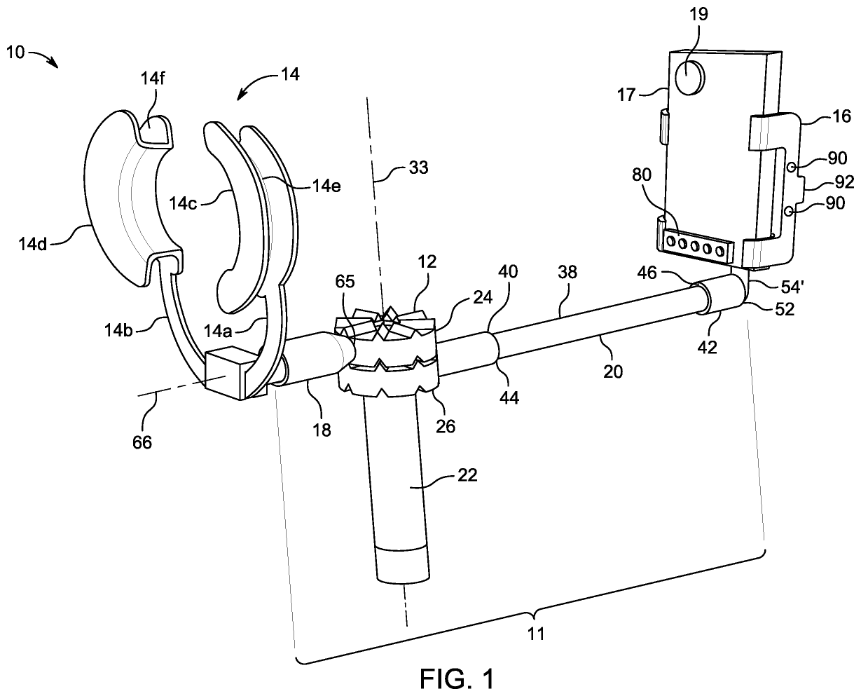

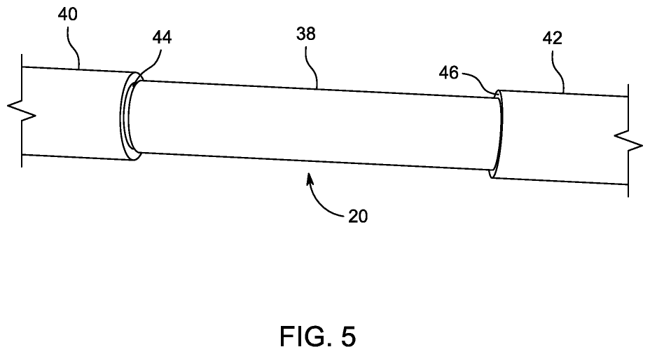

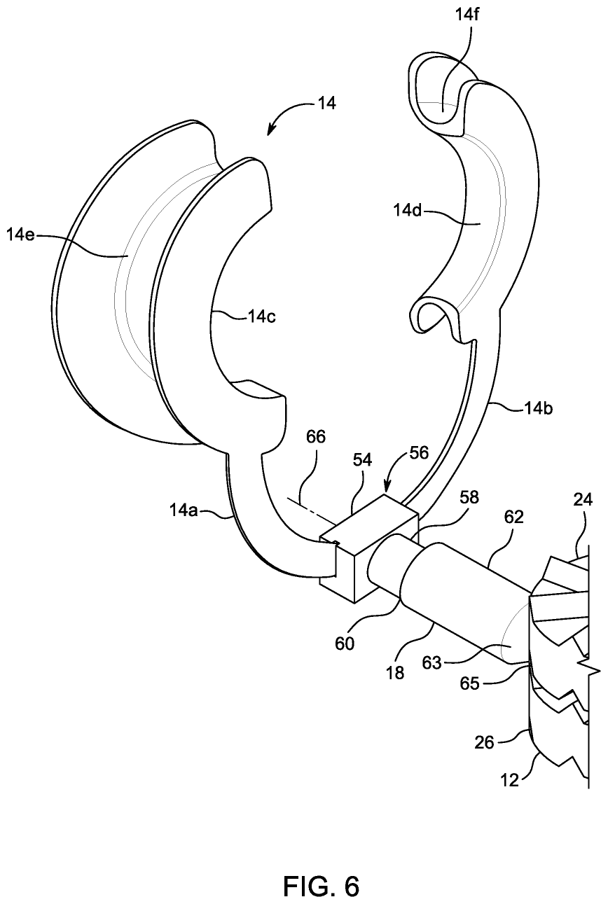

A Toothie Stick device 10 (also referred to herein as “device 10’) according to an example embodiment herein is shown in FIG. 1, and, generally (either in whole or in part) in FIGS. 1-10. The device 10 comprises at least one coupling mechanism 11, a cheek stretcher 14, a holder 16, and a handle 22. In one example embodiment herein, the coupling mechanism 11 comprises a ratcheting mechanism 12, a first connecting mechanism 18, and a second connecting mechanism 20. The cheek stretcher 14 (also referred to herein as a “mouth stretcher”) is mechanically coupled to a first end of the first connecting mechanism 18 (in one example embodiment, this is by way of a connecting component 56 (FIG. 6)), and a second, opposite end of the first connecting mechanism 18 is mechanically coupled to the ratcheting mechanism 12. As such, the cheek stretcher 14 is mechanically coupled to the ratcheting mechanism 12 by way of the first connecting mechanism 18 and connecting component 56. A first end of the second coupling mechanism 20 is mechanically coupled to the ratcheting mechanism 12, and a second, opposite end of the second coupling mechanism 20 is mechanically coupled to the holder 16. As such, the holder 16 is mechanically coupled to the ratcheting mechanism 12 by way of the second coupling mechanism 20.

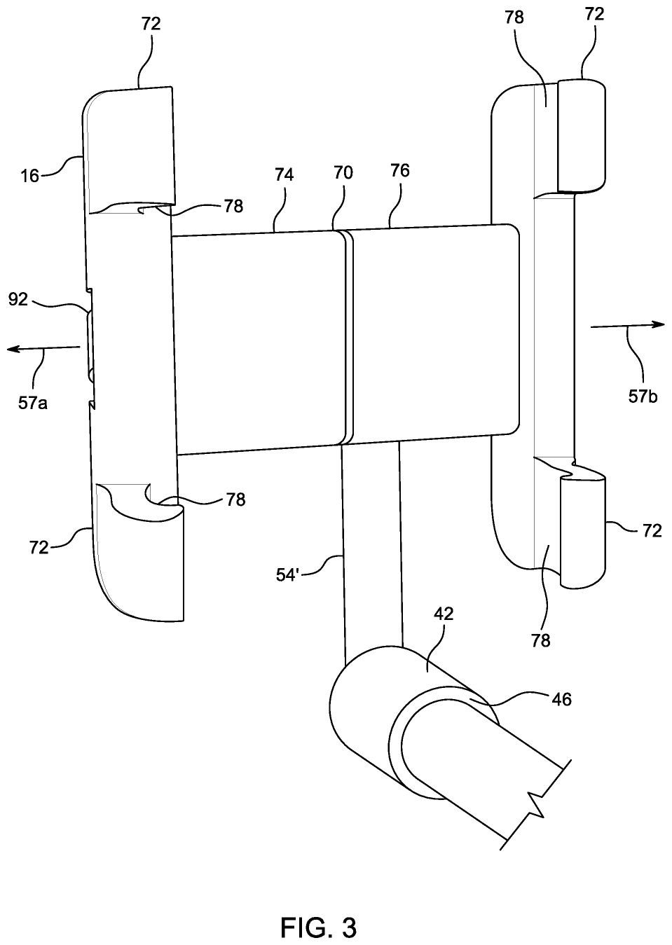

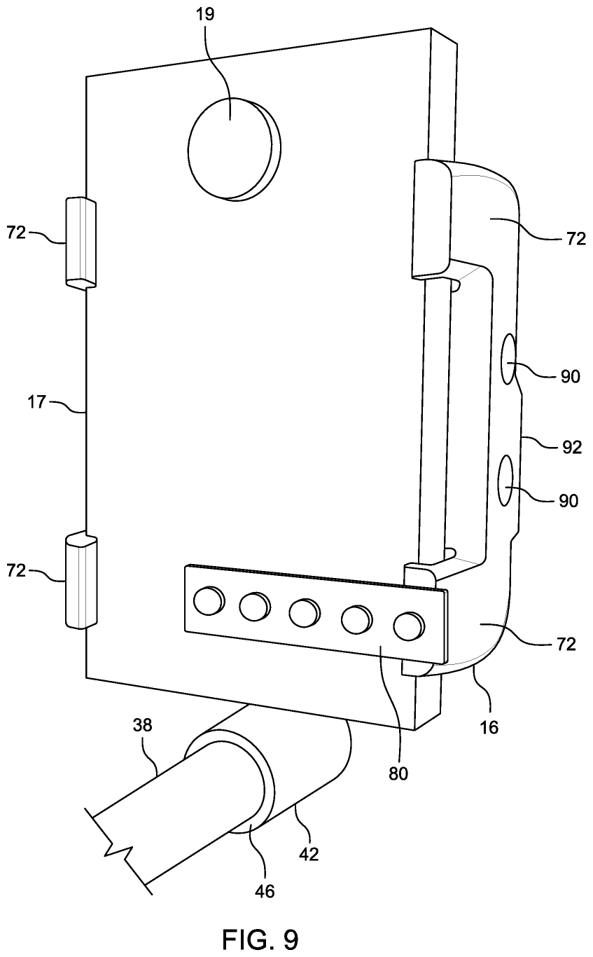

The holder 16 comprises a base 70, a plurality of grips 72, and stem 54′ (FIG. 3). An inside portion of each of the grips 72 forms a grooved seat 78 for receiving an image capturing device 17 (FIG. 1), such as a smartphone or other image capturing device. In one example embodiment herein, the base 70 and grips 72 are integrally formed and are sized to receive an image capturing device 17 of a predetermined size and shape, such that the device 17 can be removably attached within the holder 16, and such that, when attached, the device 17 maintains a substantially secure and stationary position in the holder 16. In another example embodiment herein, at least one of the base 70 and/or grips 72 is adjustable for expansion to enable the image capturing device 17 to be inserted into the holder 16, and at least one of the base 70 and/or grips 72 also is adjustable for then being compressed for securing the device 17 in the holder 16. For example, in one example embodiment the base 70 has a first portion 74 and a second portion 76 wherein those portions 74, 76 are separatably or removably attached by way of a securing/separating mechanism (e.g., a connecting track or other mechanism) (not shown). Thus, those portions 74, 76 can be attached and also can be separated by way of a mechanism, such as, e.g., a connecting track. In one example embodiment herein, the mechanism (and/or connecting track) can include one or more (e.g., plastic) notches 92 and/or one or more holes 90, and moreover in one example embodiment, one or more nuts, bolts, locking pins, springs, screws, string, wire, or the like can be used in the holes 90 and/or notches 92 to clamp the portions 74, 76 together. Expansion or opening of the holder 16 can be achieved by separating the portions 74, 76 along directions 57a, 57b, respectively, for enabling the image capturing device 17 to be received, and compression or closing of the holder 16 can be achieved by closing the parts 74, 76 back together again (along the same, but opposite directions) for securing the device 17 in the holder 16. In one example embodiment herein, one or more of the portions 74, 76 can be rotated relative the other of the portions 74, 76. In one example embodiment herein, the mechanism includes one or more nuts, bolts, locking pins, and/or springs that enable the portions 74, 76 to be separated (e.g., by pulling them apart) and rotated relative to one another. Also, in one example embodiment herein, the holder 16 can include a light source 80 such as, e.g., a light emitting diode (LED) strip or another light source (FIGS. 1 and 9). The light source 80 can be attached to, by example and without limitation, at least one of the grips 72, by way of any suitable securing mechanism. The light source 80 provides additional illumination towards the mouth stretcher 14 and for photographs taken of a patient's teeth/bite. Although not shown for convenience, the light source 80 can be powered via battery or another source of electrical power.

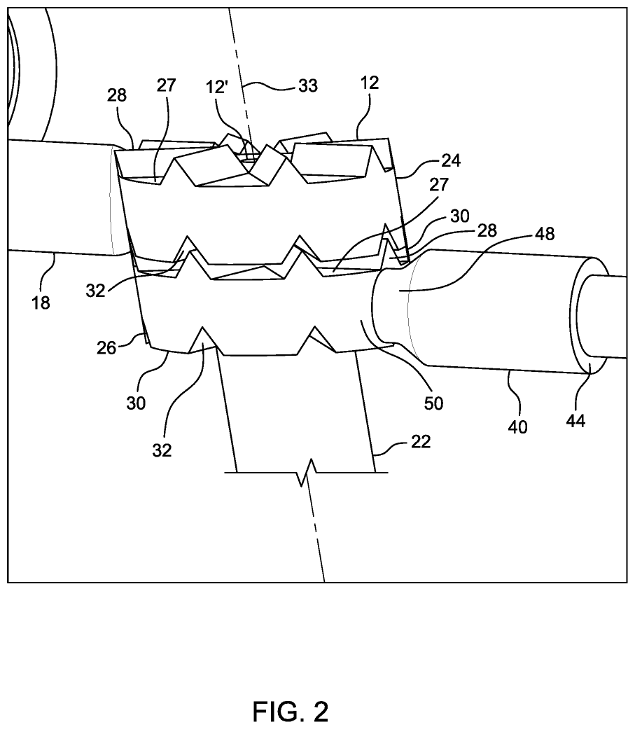

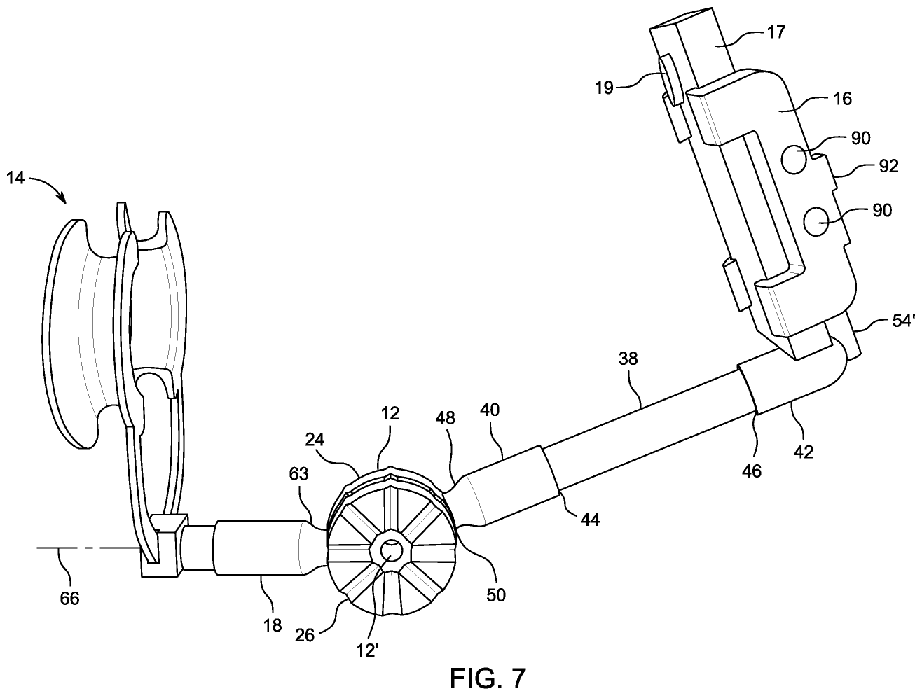

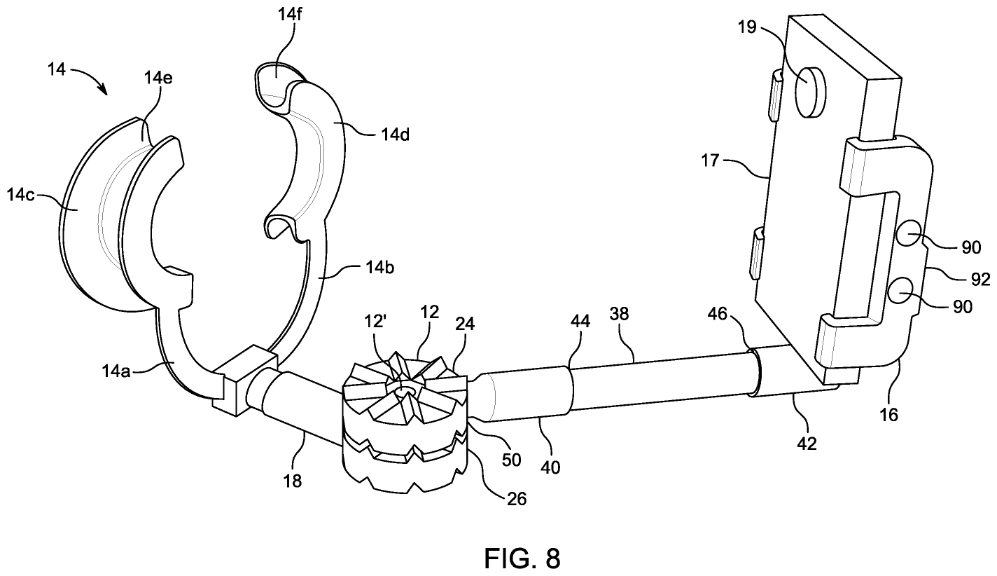

In one example embodiment herein, the ratcheting mechanism 12 preferably has a structure that ensures that the device 17 can be positioned at one of a plurality of precise angles, that cater to enabling photographs to be taken that meet predetermined standards, and which therefore minimize or reduce the number of rejected photographs. The ratcheting mechanism 12 comprises a plurality of portions, such as upper and lower portions 24, 26, respectively, each having an upper surface 27 having grooved teeth 28 (FIG. 2), and a lower surface 30 having grooves 32 shaped to receive grooved teeth 28 therein. In the illustrated embodiments, and referring in particular to FIG. 2, the grooved teeth 28 of the lower portion 26 are receivable in corresponding grooves 32 of the upper portion 24. When the grooved teeth 28 are received in the grooves 32 such the upper portion 24 sits on top of the lower portion 26 while the grooved teeth 28 of the lower portion 26 engage with the grooves 32 of the upper portion 24, the upper and lower portions 24, 26 remain in a fixed position relative to one another. When the upper and lower portions 24, 26 are separated from one another such that the grooved teeth 28 of the lower portion 26 become sufficiently disengaged with the grooves 32 of the upper portion 24, then one or each of those portions 24, 26 can be rotated with respect to the other portion, around an imaginary axis 33 passing through a center (bore hole 12′) of each portion 24, 26. By example only, during such disengagement, the first connecting mechanism 18, cheek stretcher 14, connecting component 56, and the upper portion 24 to which the cheek stretcher 14 is coupled by way of mechanism 18 and component 56, can be rotated about the axis 33 with respect to the second connecting mechanism 20, holder 16 (and any device 17 attached thereto), lower portion 26, and handle 22, and vice versa. Similarly during disengagement, the lower portion 26, second connecting mechanism 20, and holder 16 (and any device 17 attached thereto) can be rotated about the axis 33 with respect to the check stretcher 14, the first connecting mechanism 18, component 56, the upper portion 24, and the handle 22, and vice versa. FIGS. 7 and 8 represent different example rotated positions of the upper and lower portions 24, 26 relative to one another, and how the other components of device 10 are relatively orientated as a result of such positioning.

In one example embodiment herein, the second connecting mechanism 20 comprises a rod 38 having first and second ends. The first end of the rod 38 is mechanically coupled to the ratcheting mechanism 12 by way of a mechanical coupler 40, and the second end of the rod 38 is mechanically coupled to the holder 16 by way of a mechanical coupler 42. In one example embodiment, each of the couplers 40, 42 has an opening 44, 46, respectively (FIGS. 1 and 2), that extends at least partially through the respective coupler. The opening 44 of coupler 40 receives the first end of the rod 38. Also in the illustrated embodiment, an opposite end 48 of coupler 40 can be inserted into an opening in the ratcheting mechanism 12, such as an opening 50 in the lower portion 26 as represented in the illustrated embodiment (FIG. 2). Also in one example embodiment, the opening 46 of coupler 42 receives the second end of the rod 38. Also in the illustrated embodiment, an opposite end 52 of coupler 42 receives an arm 54 of holder 16.

In one example embodiment herein, the opposite end 48 of coupler 40 is at least one of removably or rotatably mechanical coupled in the opening 50 of lower portion 26, although in other examples such coupling can be a rigid mechanical coupling.

In one example embodiment herein, the arm 54 of holder 16 is at least one of removably or rotatably coupled to the coupler 42, such that, in the case of rotatable coupling, the arm 54, holder 16 (and device 17) can be rotated about an axis defined along a length of the rod 38, relative to coupler 42, and/or vice versa.

In one example embodiment herein, the first end of rod 38 is at least one of removably or rotatably coupled to the coupler 40, and/or the second end of rod 38 is at least one of removably or rotatably coupled to the coupler 42, such that, in the case of either rotatable coupling, the rod 38 and/or couplers 40, 42 (and, if desired, the components coupled thereto) can be rotated about the length of the rod 38, relative to one another.

In another example embodiment herein, the first end of rod 38 is securely and rigidly mechanically coupled to the coupler 40 (e.g., by threading or another securing mechanism), and maintains a fixed position relative to the coupler 40, and/or the second end of rod 38 is securely and rigidly mechanically coupled to the coupler 42 (e.g., by threading or another securing mechanism), and maintains a fixed position relative to the coupler 42.

In one example embodiment herein, the rod 38 has a fixed, predetermined length, and the first connecting mechanism 18 and second connecting mechanism 20 preferably have predetermined lengths that ensure that the teeth stretcher 14 and device 17 are positioned at a predetermined distance away from one another, and also to ensure that the device 17 is held at a predetermined distance relative a mouth of a patient. In another example embodiment, at least one of the first connecting mechanism 18 or the second connecting mechanism 20 is telescopic in nature or otherwise has an adjustable length, such that, by adjusting the length(s), positions of the holder 16 (and device 17) and/or the mouth stretcher 14 relative to one another also can be adjusted as desired.

In the illustrated example embodiment, and referring to FIG. 6, the cheek stretcher 14 comprises two curved portions 14a, 14b that are connected (rotatably in one example, or rigidly in another example) at first ends thereof to a base 54 of connecting component 56 such that the curved portions 14a, 14b and base 54 collectively form an arc-type shape. The connecting component 56 also comprises a stem 58 that extends into an opening 60 of a coupler 62 (which forms at least part of first connecting mechanism 18). In one example embodiment herein, the stem 58 is removably and rotatably attached to the coupler 62, such that the overall stretcher 14 can be rotated relative to an imaginary axis 66 defined by and extending through a length of the coupler 62 and connecting component 56. In another example embodiment herein the stem 58 is securely and rigidly mechanically coupled to the coupler 62 (e.g., by threading or another securing mechanism), and maintains a fixed position relative to the coupler 62.

Attached at a second end of the curved portion 14a is a first mouthpiece 14c that is curved in shape and which includes an annular groove 14e for receiving lips (and cheeks) of one side of a patient's mouth. Attached at a second end of the curved portion 14b is a second mouthpiece 14d that is curved in shape and which includes an annular groove 14f for receiving lips (and cheeks) of an opposite side of the patient's mouth. When the curved portions 14c and 14d are positioned in the patient's mouth, together they enable the patient's lips to be opened to an extent that enables at least a substantial portion of the patient's teeth/bite to be exposed to a camera 19 of the device 17, for being captured in an image. The cheek stretcher 14 ensures the user's cheeks are out of the way when taking pictures of the user's teeth. By virtue of the configuration of the overall device 10 wherein the mouth stretcher 14 is positioned at a controlled distance from the device 17, images of the patient's teeth can be captured which are less susceptible to being rejected for use in creating dental aligners.

Although the curved portions 14a, 14b in one example embodiment can be rigidly connected to the base 54 of connecting component 56, in another example embodiment herein the curved portions 14a, 14b are at least one of removably or rotatably coupled to the base 54 such that, in the case of rotatable coupling, the curved portions 14a, 14b and mouth pieces 14c, 14d can be rotated relative to imaginary axis 66. For example, in one embodiment, the portions 14a, 14b are integrally formed in a single structure that is rotatably coupled to the base 54. By example, the integrally formed structure can be inserted into a slot in the base 54 so the structure becomes coupled thereto in a manner that enables the structure to be rotated relative to the base 54, as represented in 1208 of FIG. 12 (to be described below).

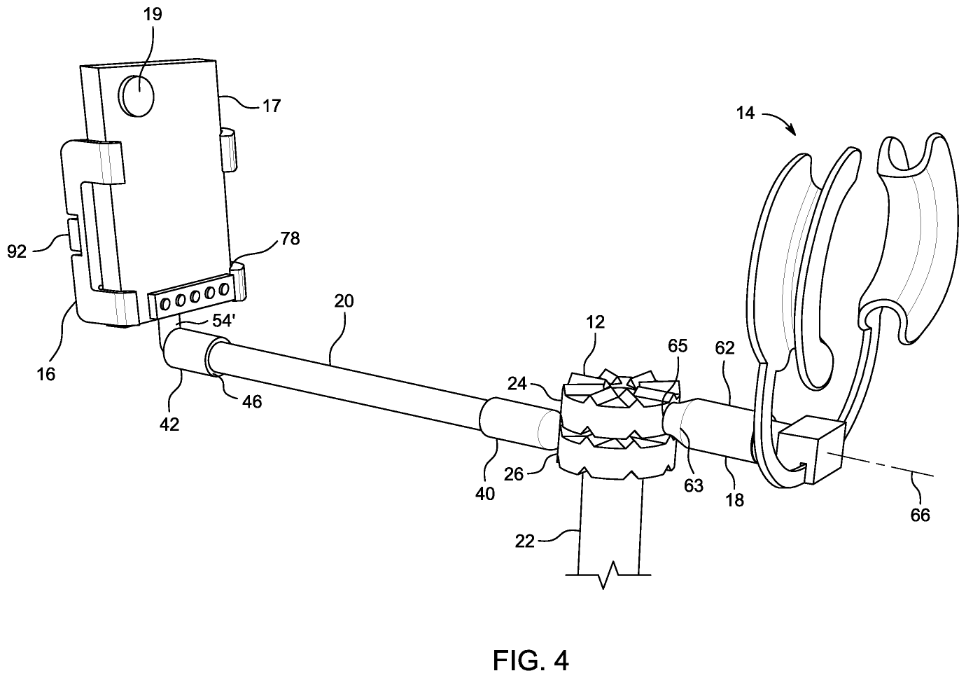

Referring again to coupler 62, an end 63 of coupler 62 opposite the opening 60 can be inserted into an opening in the ratcheting mechanism 12, such as an opening 65 in the upper portion 24 of the mechanism 12, as represented in the illustrated embodiment (FIG. 4). In one example embodiment herein, the end 63 of coupler 62 is at least one of removably or rotatably attached/coupled in the opening 65 of ratcheting mechanism 12, such that, in the case of rotatable attachment, the overall cheek stretcher 14 (and connecting component 56) can be rotated with respect to axis 66. In another example embodiment herein, the coupler 62 is securely and rigidly mechanically coupled with the opening 65 of the ratcheting mechanism 12 (e.g., by threading or another securing mechanism), and maintains a fixed position relative to the mechanism 12.

Similarly, referring again to coupler 40, end 48 (FIG. 2) thereof is at least one of removably or rotatably attached/coupled in opening 50 of ratcheting mechanism 12 (lower portion 26), such that, in the case of rotatable attachment, the coupler 40 (and rod 38, coupler 42, holder 16, and device 17) can be rotated about an axis defined along a length of the rod 38, relative to the ratcheting mechanism 12, and/or vice versa. In another example embodiment herein, the coupler 40 is securely and rigidly mechanically coupled with the opening 50 of the ratcheting mechanism 12 (e.g., by threading or another securing mechanism), and maintains a fixed position relative to the mechanism 12.

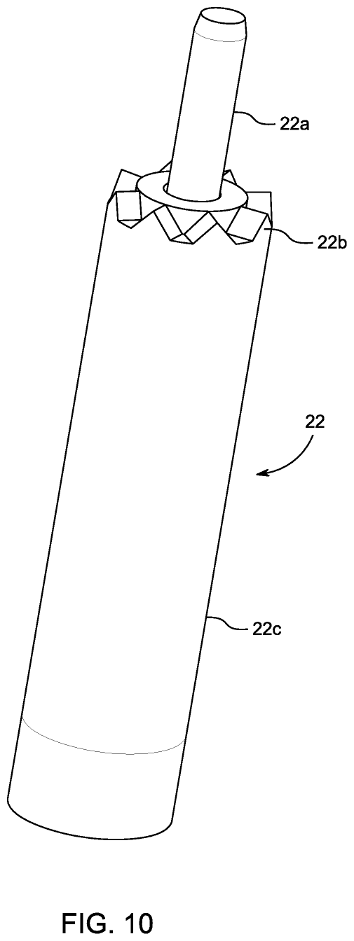

Also in one example embodiment herein, the handle 22 is at least one of removably or rotatably attached/coupled to the lower portion 26 of ratcheting mechanism 12 such that, in the case of rotatable attachment/coupling, the lower portion 26 and/or handle 22 can be rotated related to one another. FIG. 10 shows one example embodiment of the handle 22 in more detail. In the illustrated embodiment, the handle 22 includes an elongate portion 22c, a spindle 22a for being inserted into a bore hole 12′ (FIGS. 7 and 8) in the ratcheting mechanism 12, and grooved teeth 22b that are arranged circumferentially at an end of the elongate portion 22c adjacent the spindle 22a. In one example embodiment herein, the elongate portion 22c and spindle 22a are integrally formed, although in other example embodiments they may be separate parts, and/or may be removably attached to one another.

Also in one example embodiment herein, and although not shown for convenience, the bore hole 12′ is formed in the lower portion 26 of the mechanism 12, or, in another example embodiment herein, in both the lower portion 26 and the upper portion 24 of the mechanism 12. The spindle 22a can be received in the bore hole 12′ preferably to an extent such that lower surface 30 (FIG. 2) of the lower portion 26 engages with the teeth 22b of the handle 22. In one example embodiment herein, the lower surface 30 of the lower portion 26 includes a teethed surface having grooves 32 with which the teeth 22b of the handle 22 can be engaged. When both surfaces are so engaged, the handle 22 and the ratcheting mechanism 12 maintain a substantially fixed and secured position relative to one another. When removed from such engagement, one or both of the ratcheting mechanism 12 and handle 22 can be rotated about imaginary axis 33, and then the ratcheting mechanism 12 and handle 22 can be re-engaged such that they maintain a new relative orientation with respect to one another, in a fixed and secured manner.

In another example embodiment herein, the handle 22 is securely and rigidly mechanically coupled to the ratcheting mechanism 12 (e.g., by threading or another securing mechanism), and maintains a fixed position/orientation relative to the mechanism 12.

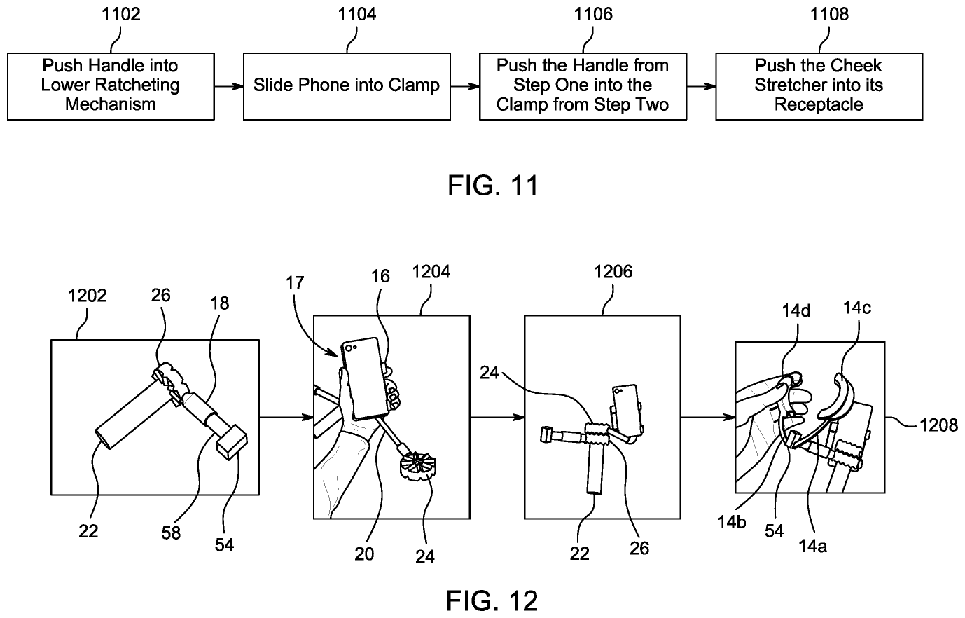

Methods according to example embodiments herein will now be described. FIG. 11 shows a flow diagram of a method for assembling a Toothie Stick (e.g., such as device 10), and FIG. 12 represents assembly of the Toothie Stick according to that method, in an example embodiment herein. In steps 1100 and 1202 the handle 22 is inserted into the lower portion 26. By example, the spindle 22a is inserted into bore hole 12′ the lower portion 26 as described above. In step 1104 an image capturing device 17 (e.g., a smartphone) is clamped in the holder 16, wherein the holder 16 is shown in step 1204 as being attached to upper portion 24 by way of second connecting mechanism 20. In steps 1106 and 1206 the upper portion 24 and lower portion 26 are engaged with one another to form the ratcheting mechanism 12. In one example embodiment herein, such engagement includes engaging grooved teeth 28 of upper surface 27 of lower portion 26 with corresponding grooves 32 of the upper portion 24, and, in one example in which the spindle 22a is of sufficient length and the bore hole 12′ also is formed in upper portion 24, the engagement also can include inserting the spindle 22a into the portion of bore hole 12′ included in the upper portion 24. In steps 1108 and 1208 the curved portions 14a, 14b (which are shown connected to mouthpieces 14c, 14d) are inserted into the base 54 to form teeth stretcher 14. In one example embodiment herein, and as represented in step 1208 of FIG. 12, the curved portions 14a, 14b are an integrally formed structure that is inserted into a groove or slot of the base 54, such that the structure becomes coupled to the base 54 in a rotatable manner relative to the base 54, although in other examples the structure is coupled to the base in a rigid and/or non-rotatable manner. In one example embodiment herein, the structure is detachably coupled to the base 54. The foregoing is one example of a manner in which the Toothie Stick 10 is assembled. Of course, this example is not limiting, and the Toothie Stick 10 can be assembled in other manners such as with components thereof being connected in a different order than explained above.

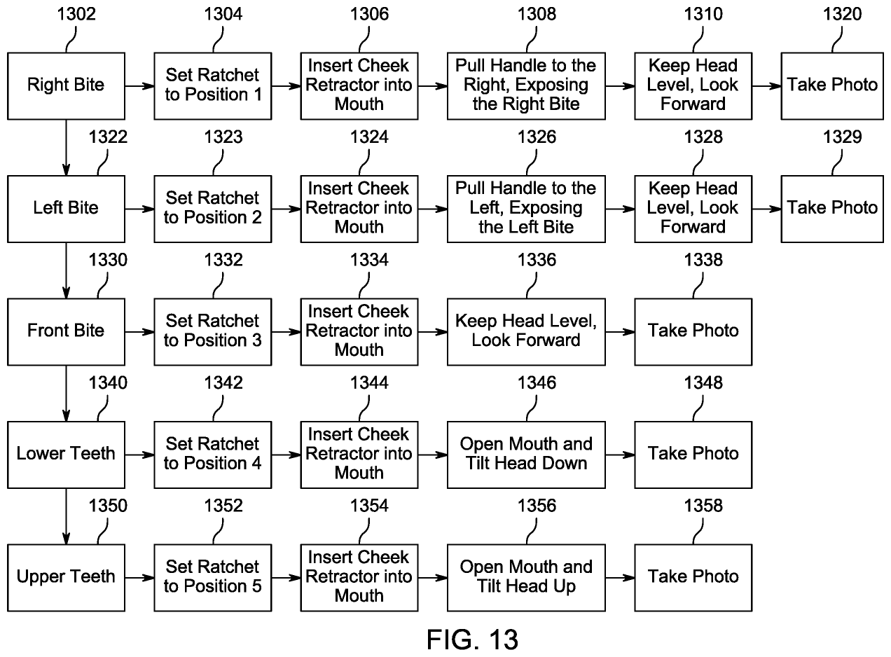

An example method for using a Toothie Stick 10 according to an example aspect herein will now be described, with reference to FIG. 13. To obtain a photograph of a right bite 1302 of a patient's teeth, the ratcheting mechanism 12 of the Toothie Stick 10 is set to a first position (e.g., the upper and lower portions 24, 26 of ratcheting mechanism 12 are positioned relative to one another in a first predetermined orientation for capturing the right bite 1302) in step 1304. In step 1306 the mouthpieces 14c, 14d (forming a teeth retractor) are inserted into the patient's mouth. The patient can then pull the handle 22 towards his/her right, thereby exposing the right bite (step 1308). The patient then maintains his/her head level and looks forward in step 1310. Then, in step 1320, the photograph is taken of the exposed bite, using, for example, the image capturing device 17, which is optimally positioned to capture an image of the right bite by virtue of the foregoing steps.

To obtain a photograph of a left bite 1322 of the patient's teeth, the ratcheting mechanism 12 of the Toothie Stick is set to a second position (e.g., the upper and lower portions 24, 26 are positioned relative to one another in a second predetermined orientation for capturing the left bite 1322) in step 1323. In step 1324 the mouthpieces 14c, 14d (forming a teeth retractor) are inserted into the patient's mouth. The patient can then pull the handle 22 towards his/her left, thereby exposing the right bite (step 1326). The patient then maintains its head level and looks forward in step 1328. Then, in step 1329, the photograph is taken of the exposed bite, using, for example, the image capturing device 17, which is optimally positioned to capture an image of the right bite by virtue of the foregoing steps.

To obtain a photograph of a front bite 1330 of the patient's teeth, the ratcheting mechanism 12 of the Toothie Stick is set to a third position (e.g., the upper and lower portions 24, 26 are positioned relative to one another in a third predetermined orientation for capturing the front bite 1330) in step 1332. In step 1334 the mouthpieces 14c, 14d (forming a teeth retractor) are inserted into the patient's mouth. The patient can then maintain his/her head level, and look forward, exposing the front bite (step 1336). Then, in step 1338, the photograph is taken of the exposed front bite, using, for example, the image capturing device 17, which is optimally positioned to capture an image of the front bite by virtue of the foregoing steps.

To obtain a photograph of the patient's lower teeth (1340), the ratcheting mechanism 12 of the Toothie Stick 10 is set to a fourth position (e.g., the upper and lower portions 24, 26 are positioned relative to one another in a fourth predetermined orientation for capturing lower teeth) in step 1342. In step 1344 the mouthpieces 14c, 14d (forming a teeth retractor) are inserted into the patient's mouth. The patient can then open its mouth and tilt its head downwardly (step 1346). Then, in step 1348, the photograph is taken of the lower teeth, using, for example, the image capturing device 17, which is optimally positioned to capture an image of the lower teeth by virtue of the foregoing steps.

To obtain a photograph of the patient's upper teeth (1350), the ratcheting mechanism 12 of the Toothie Stick 10 is set to a fifth position (e.g., the upper and lower portions 24, 26 are positioned relative to one another in a fifth predetermined orientation for capturing upper teeth) in step 1352. In step 1354 the mouthpieces 14c, 14d (forming a teeth retractor) are inserted into the patient's mouth. The patient can then open their mouth and tilt their head upward (step 1356). Then, in step 1358, the photograph is taken of the upper teeth, using, for example, the image capturing device 17, which is optimally positioned to capture an image of the upper teeth by virtue of the foregoing steps.

The components of the Toothie Stick 10 can be made from any suitable material(s), including, without limitation and by example, plastic. The Toothie Stick 10 can be used to control the orientation, position, and angle of a camera device 17 relative to one's mouth, to enable capture of high quality images that can be used for creating a dental aligner.

While various example embodiments of the present invention have been described above, it should be understood that they have been presented by way of example, and not limitation. It will be apparent to persons skilled in the relevant art(s) that various changes in form and detail can be made therein. Thus, the present invention should not be limited by any of the above described example embodiments, but should be defined only in accordance with the following claims and their equivalents.

In addition, it should be understood that the Figures are presented for example purposes only. The architecture of the example embodiments presented herein is sufficiently flexible and configurable, such that it may be utilized (and navigated) in ways other than that shown in the accompanying figures.

Further, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the example embodiments presented herein in any way. It is also to be understood that the procedures recited in the claims need not be performed in the order presented.

What is claimed is:

1. A camera stick, comprising:
a dental component for being positioned in a subject's mouth;
a holder for holding a camera device; and
at least one coupling mechanism mechanically coupled to the dental component and the holder, the at least one coupling mechanism being adjustable for enabling the dental component and holder to be positioned in space at a selected position and orientation relative to one another.

2. The camera stick of claim 1, wherein the at least one coupling mechanism comprises:
a first coupling mechanism;
a second coupling mechanism; and
a ratcheting mechanism interposed between, and mechanically coupled to, the first and second coupling mechanisms,
wherein the ratcheting mechanism enables at least one of the first and second coupling mechanisms to be rotated relative to another one of the first and second coupling mechanisms.

3. The camera stick of claim 1, wherein the dental component comprises a cheek stretcher.

4. The camera stick of claim 3, wherein the camera stick also comprises a connecting component mechanically coupling the cheek stretcher to the at least one coupling mechanism.

5. The camera stick of claim 2, wherein the dental component comprises a cheek stretcher, and the camera stick further comprises a connecting component having a base, the cheek stretcher being rotatably mechanically coupled to the base, the connecting component mechanically rotatably coupled to the first coupling mechanism.

6. The camera stick of claim 1, wherein at least one of the holder and the dental component is mechanically rotatably coupled to the coupling mechanism.

7. The camera stick of claim 3, wherein the cheek stretcher comprises a first mouthpiece and a second mouthpiece, each being curved in shape and including an annular groove for receiving the subject's mouth.

8. The camera stick of claim 7, wherein the cheek stretcher enables the subject's mouth to be opened to an extent that enables at least a substantial portion of at least one of a bite or teeth of the subject's mouth to be exposed to the camera device for being captured in an image.

9. The camera stick of claim 2, wherein the ratcheting mechanism comprises an upper portion and a lower portion that are rotatably engaged with one another.

10. The camera stick of claim 9, wherein the first coupling mechanism is rotatably mechanically coupled to the upper portion, and the second coupling mechanism is rotatably mechanically coupled to the lower portion.

11. The camera stick of claim 2, further comprising a handle mechanically coupled to the ratcheting mechanism.

12. The camera stick of claim 10, wherein the upper portion includes an upper surface and a lower surface, the lower portion also includes an upper surface and a lower surface, and the upper surface of the lower portion is engaged with the lower surface of the upper portion.

13. The camera stick of claim 12, wherein at least one of the upper surface or the lower surface of at least one of the upper portion or the lower portion forms a teethed surface.

14. The camera stick of claim 12, further comprising a handle rotatably mechanically coupled to the lower surface of the lower portion.

15. The camera stick of claim 2, wherein at least one of the first coupling mechanism or the second coupling mechanism is rotatable around an axis extending along a length of the at least one of the first coupling mechanism or the second coupling mechanism.

16. The camera stick of claim 11, wherein the handle includes an elongate portion and a spindle attached thereto, the spindle being insertable into a bore hole of the ratcheting mechanism.

17. A method of forming a camera stick, comprising:
inserting a handle into an opening of a lower portion of a ratcheting mechanism, the lower portion being mechanically coupled to a first end of a first coupling mechanism;
engaging an upper portion of the ratcheting mechanism with the lower portion, the upper portion coupled to a holder for holding a camera device; and
coupling a cheek stretcher to a second end of the first coupling mechanism.

18. A method of operating a camera stick, comprising:
setting a ratcheting mechanism of the camera stick to a predetermined position, the camera stick including the ratcheting mechanism, a cheek stretcher coupled to the ratcheting mechanism by way of a first coupling mechanism, a camera holder coupled to the ratcheting mechanism by way of a second coupling mechanism, and a handle coupled to a lower surface of the ratcheting mechanism;
inserting the cheek stretcher into a subject's mouth; and
operating a camera device held by the camera holder to take a picture of a predetermined part of the subject's mouth.

19. The method of claim 18, further comprising:
positioning the handle relative to the cheek stretcher to cause the predetermined part of the subjectt's mouth to be exposed to a view of the camera device.

20. The method of claim 18, wherein the predetermined part of the subject's mouth includes at least one of a right bite, a left bite, a front bite, lower teeth, and upper teeth of the subject.
